(19) United States
(12) Patent Application Publication
(10) Pub. No.: US 2026/0327564 A1
(43) Pub. Date: Oct. 22, 2026

(54) THREE-DIMENSIONAL (3D) BIO-PRINTED CARTILAGE ORGANOIDS AND METHOD FOR GENERATING THE SAME

(71) Applicant: Center for Neuromusculoskeletal Restorative Medicine Limited, Pak Shek Kok (HK)

(72) Inventors: Chao WAN, Shatin (HK); Hongji ZHANG, Shatin (HK); Fengjie ZHANG, Shatin (HK)

(21) Appl. No.: 19/550,451

(22) Filed: Feb. 26, 2026

Related U.S. Application Data
(60) Provisional application No. 63/791,948, filed on Apr. 21, 2025.

(51) Int. Cl. C12N 5/077; B33Y 40/10; B33Y 70/10; B33Y 80/00

(52) U.S. Cl. CPC C12N 5/0655; B33Y 40/10; B33Y 70/10; B33Y 80/00; C12N 2503/04; C12N 2506/1392; C12N 2513/00; C12N 2533/30; C12N 2533/74; C12N 2537/10

(57) ABSTRACT

A method for generating three-dimensional (3D) cartilage organoids through bioprinting, the method comprising: expanding mesenchymal stem cells (MSCs); preparing a composite bioink; and, bioprinting a cell-laden scaffold to generate the 3D cartilage organoids. Also disclosed is a three-dimensional (3D) cartilage organoid for use in disease modeling and screening, the organoid comprising: human multipotent mesenchymal stem cells (MSCs) and a composite bioink.

### CROSS-REFERENCE TO RELATED APPLICATIONS

The present application claims priority to U.S. Provisional Application No. 63/791,948, filed on Apr. 21, 2025.

### FIELD OF THE INVENTION

The present invention relates to a field of tissue engineering for disease modeling and drug screening. More particularly, the present invention pertains to a three-dimensional (3D) bio-printed cartilage organoids and method for generating the same.

### BACKGROUND OF THE INVENTION

Traditional in vitro two-dimensional (2D) cell cultures and in vivo animal models have been extensively utilized in joint disease research, as well as in the fields of disease modeling and drug screening. While 2D cultures are cost-effective, highly reproducible, and convenient, with minimal resource requirements and the ability to support high-throughput testing, they are limited by their oversimplified nature. These models fail to replicate a 3D microenvironment found in vivo, and the physical and chemical properties of their substrates, typically polystyrene plastic, differ significantly from the native extracellular matrix (ECM) of joint tissues. As a result, 2D models are generally used for preliminary experiments before transitioning to more complex and physiologically relevant models for in-depth studies.

Native joint tissues are exposed to a complex interplay of biomolecular signals and physical loading, which are difficult to replicate in current in vitro 2D cell cultures models. As a result, animal models have been widely used for decades to study joint diseases, significantly advancing the understanding of osteoarthritis, including its onset, mechanisms, molecular genetics, environmental influences, and the safety and efficacy of therapies. Unlike conventional 2D cell culture models, animal models enable the study of changes across all joint components and their interactions during disease progression. However, significant anatomical, biomechanical, and genetic differences between animal models and humans often limit the translation of findings from animal studies to clinical applications.

Further, articular cartilage is characterized by its avascular, aneural structure and lack of lymphatic drainage, which limits its capacity for self-regeneration. Current cartilage regeneration methods primarily focus on alleviating joint pain and improving functionality, but these traditional treatments often lack long-term effectiveness. Non-steroidal anti-inflammatory drugs (NSAIDs), such as Celecoxib and Diclofenac, are commonly used to alleviate joint pain and inflammation associate with osteoarthritis by inhibiting cyclooxygenase (COX) isoenzymes. This inhibition reduces the production of prostanoids, providing both anti-inflammatory and analgesic effects. However, the use od NSAIDs carries significant risks, contributing to approximately 30% of hospital admissions due to adverse drug reactions. These risks include severe gastrointestinal issues, an increased likelihood of cardiovascular disease, and renal failure. For elderly patients, the long-term use of NSAIDs may post risks that outweigh the potential benefits in managing osteoarthritis. Matrix-assisted chondrocyte implantation is used for treatment of cartilage lesions, but the insufficient biomechanical properties may cause side effects such as cartilage fibrosis or hyperthrophy.

Therefore, there is a critical clinical need for innovative therapies aimed at cartilage repair and regeneration. Cartilage tissue engineering has emerged as a promising solution to regenerate articular cartilage tissue damaged by degeneration or injury. Recently, 3D bioprinting has emerged as a viable alternative to address these challenges. This technology allows for the precise printing of cells and biomaterials into predesigned spatial configurations, enabling the creation of complex, three-dimensional structures with high accuracy and reproducibility. However, current engineered solutions face limitations in fully repairing articular cartilage damage, particularly due to challenges in cell-biomaterial interactions, biocompatibility, and tissue integration. A major obstacle is the difficulty of replicating the native structure and function of cartilage using traditional methods.

Several 3D bioprinting methods and systems exist in the market. However, no comprehensive solution has been developed to fully address the above issues. Some of these examples are discussed in the following prior arts.

WIPO patent publication no. 2022261534A2 relates to three-dimensional, cell-laden bioink scaffolds, methods of making and using the same. A bioink for rapid fabrication of cell-loaded 3D constructs at subzero temperatures. The bioink allows the fabrication of multilayered structures at low extrusion pressure without interlayer mixing and causing cell death. Said prior art also creates a 3D construct using an alginate-collagen-based bioink loaded with mesenchymal stem cells (MSCs). The prior art further provides a method of making a frozen, three-dimensional, cell-laden bioink scaffold. However, said 3D bioink scaffolds may have low breaking stress and the ability to self-recover.

United States patent publication no. 20230293306A1 relates to a bionic tissue stent, a preparation method therefor and an application thereof. The preparation method of said invention is a 3D printing demolding method. Said invention further provides a method for preparing a bionic tissue scaffold by 3D printing a sacrificial material to form a mold, pouring a hydrogel composition into the mold for in-situ crosslinking and curing to obtain a hydrogel-mold complex, dissolving the mold using a solvent to obtain a cured hydrogel, and freeze-drying the cured hydrogel to produce the bionic tissue scaffold. However, said scaffolds may have low breaking stress and the ability to self-recover.

Several studies have explored methods for producing bioink for 3D bioprinting systems. For example, Gungor-Ozkerim et al. (2018) provides an in-depth overview of the state of the art in existing bioinks. The review covers both natural and synthetic biomaterials, used either alone or in combination, and, in some cases, multicellular spheroids used as bioinks. Studies that involve printing biopolymers without embedded cells or seeding cells post-printing are excluded, focusing only on bioinks that contain cells. The bioink in the prior art exhibits high stiffness and retains its shape well after mechanical stress. However, this may not be ideal for cartilage repair and regeneration, as cartilage requires a balance between stiffness and viscoelasticity to mimic its natural properties. Excessive rigidity could hinder cell proliferation and extracellular matrix (ECM) deposition, which are both essential for functional tissue development.

Despite advancements in existing technology and research in 3D bioprinting, there remains a need for improved methods that can be more effectively utilized in areas such as the development of clinically relevant disease models and high-throughput drug screening for evaluating the efficacy of various molecules and therapies. The global osteoarthritis treatment market is experiencing steady growth, driven by the rising incidence of osteoarthritis among aging populations. According to recent studies, in 2019, approximately 528 million people worldwide were living with osteoarthritis, representing a 113% increase since 1990. The prevalence of osteoarthritis is expected to rise further in the coming years, creating a substantial market demand for effective treatments. These challenges highlight the growing need for tissue engineering solutions that utilize cells and biomaterials to repair or replace damaged cartilage. Among these advancements, three-dimensional (3D) bioprinting, which leverages computer imaging and patient-specific data, is recognized as a transformative technology for producing precise and highly customized 3D cartilage organoids for cartilage tissue engineering, disease modeling and drug development.

### SUMMARY OF THE INVENTION

It is an objective of the present invention to provide 3D bio-printed cartilage organoids and a method for generating the same, capable of producing patient-specific models that accurately replicate the native cartilage's anatomical structure, enhancing disease modeling, drug screening outcomes, and therapeutic applications while leveraging patient medical imaging.

It is also an objective of the present invention to provide 3D bio-printed cartilage organoids and a method for generating the same, capable of supporting high-throughput production with minimal batch-to-batch variability, thus ensuring consistency and reliability for drug development and clinical applications.

It is further an objective of the present invention to provide 3D bio-printed cartilage organoids and a method for generating the same using a composite bioink, enabling the development of advanced drug screening models and facilitating the evaluation of various molecules and therapies in clinically relevant disease models.

A further objective of the present invention is to provide 3D bio-printed cartilage organoids and a method for generating the same using a composite bioink, improving articular cartilage reparation and regeneration where the engineered cartilage organoid integrates patient-specific cartilage structures and precisely replicate the anatomical properties of native cartilage.

Accordingly, these objectives may be achieved by following the teachings of the present invention. The present invention relates to a method for generating 3D cartilage organoids through bioprinting, the method comprising: expanding mesenchymal stem cells (MSCs); preparing a composite bioink; and, bioprinting a cell-laden scaffold to generate the 3D cartilage organoids.

Additionally, the present invention also teaches three-dimensional (3D) cartilage organoid for use in disease modelling and screening, the organoid comprising: human multipotent mesenchymal stem cells (MSCs) and a composite bioink.

### BRIEF DESCRIPTION OF DRAWINGS

The features of the invention will be more readily understood and appreciated from the following detailed description when read in conjunction with the accompanying drawings of the preferred embodiment of the present invention.

FIGS. 1A and 1B illustrate schematic diagrams of the preparation flow for 3D-printed cartilage organoids, their formation using bioprinting, and their applications in (FIG. 1A) in vivo transplantation for clinical cartilage repair, and (FIG. 1B) drug screening and joint disease modeling.

FIG. 2 illustrates a printability evaluation of different bioinks under various parameters.

FIGS. 3A-3B illustrate cell-laden scaffolds with various shapes and dimensions, wherein FIG. 3A illustrates a top view of a 10-layer cube that retains a consistent shape as its height increases, and FIG. 3B illustrates a top view of well-defined printed strands in a lattice structure.

FIGS. 4A-4B illustrate the rheological properties of different bioinks NC, PEG and ALG at room temperature, wherein FIG. 4A illustrates viscosity at a constant shear rate of 0.1 s−1. It is seen that the low viscosity of PEG is increased by the addition of ALG and further strongly increased by blending with NC, and FIG. 4B illustrates a shear thinning effect of bioinks at increasing shear rates.

FIGS. 5A-5D illustrate viscoelastic behavior of different bioinks and crosslinked hydrogel consisting of nanoclay, PEG and alginate at room temperature, wherein FIG. 5A and FIG. 5B illustrate frequency sweep measurements of different bioinks, FIG. 5C illustrates amplitude sweep measurements of different printed hydrogel ranging from 0% to 10% strain, and FIG. 5D illustrates frequency sweep measurements of different printed hydrogel ranging from 0.1 Hz to 2 Hz.

FIGS. 6A-6C illustrate DX and EDX mapping of a cross section of a printed NC-PEG-ALG bioscaffold strand, wherein FIG. 6A and FIG. 6B illustrate EDX element analysis revealing characteristic peaks of NC-PEG-ALG; FIG. 6C illustrates mapping of Si and Mg, and its merge shows distribution of nanoclay in the strand of scaffold, while element mapping of S and Si and its merge reveals integration of PEG-VS and nanoclay.

FIGS. 7A-7C illustrate EDX and EDX mapping of a cross section of a printed ALG bioscaffold strand, wherein FIG. 7A and FIG. 7B illustrate characteristic peaks of ALG, and FIG. 7C illustrates an element mapping of Ca shows the crosslinked strand of the bioscaffold.

FIG. 8 illustrates undissolved bioscaffolds treated with 100 mM citric acid.

FIG. 9 illustrates mass swelling ratio characterization of ALG hydrogel and NC-PEG-ALG scaffold.

FIG. 10 illustrates strand width change of ALG scaffold and NC-PEG-ALG scaffold.

FIG. 11 illustrates a morphology of generated strands from NC-PEG-ALG and ALG hydrogel incubated in PBS for 7 days.

FIG. 12 illustrates cell encapsulated alginate scaffold and composite scaffold incubated in culture medium at 37° C.

FIG. 13 illustrates breaking compressive stress comparison among 3D bioprinted ALG, PEG-ALG, and the NC-PEG-ALG bioscaffolds.

FIG. 14 illustrates Young's modulus comparison among 3D bioprinted ALG, PEG-ALG, and the NC-PEG ALG bioscaffolds.

FIG. 15 illustrates self-recovery profile of the NC-PEG-ALG bioscaffold.

FIGS. 16A-16H illustrate a scanning electron microscope characterization of the NC-PEG-ALG bioscaffold and ALG bioscaffold, wherein (FIGS. 16A-16D) illustrate top view and side view of the dehydrated alginate hydrogel with homogeneous appearance and low roughness, and FIGS. 16E-16H illustrate top view and side view of the freeze-dried composite hydrogel with nanoscale porous structure.

FIGS. 17A-17B illustrate cell viability analysis of the 3D bioprinted cartilage organoids, where FIG. 17A illustrates LIVE/DEAD staining for chondrocytes encapsulated in ALG cartilage organoids and the NC-PEG-ALG cartilage organoids, and FIG. 17B illustrates viability of encapsulated cells in ALG cartilage organoids and the NC-PEG-ALG cartilage organoids cultured up to 7 days.

FIGS. 18A-18B illustrate release profile of model proteins from the NC-PEG-ALG bioscaffolds and ALG bioscaffolds, wherein FIG. 18A shows the in vitro cumulative release curve of BSA from NC-PEG-ALG hydrogel and ALG hydrogel, while FIG. 18B presents the cumulative release curve of lysozyme from the same hydrogels.

FIG. 19A illustrates absorption of BSA and FIG. 19B illustrates absorption of lysozyme in NC-PEG-ALG bioscaffolds and ALG bioscaffolds.

FIGS. 20A-20E illustrate histological staining and immunostaining of chondrocytes embedded NC-PEG-ALG bioscaffolds following culture over 2 weeks, wherein representative images of (FIG. 20A) Safranin O staining, (FIG. 20B) Alcian blue staining, (FIG. 20C) H&E staining, (FIG. 20D) Anti-SOX9, (FIG. 20E) Anti-Col II immunohistochemistry staining, on cryosections of chondrocytes encapsulated NC-PEG-ALG bioscaffold at day 14.

FIGS. 21A-21J illustrate histological staining and immunostaining of human umbilical cord derived mesenchymal stem cells (hUCMSCs) embedded NC-PEG-ALG bioscaffolds following culture over 7 days and 14 days, wherein, representative images of Anti-COL II (FIGS. 21A-21B) and Anti-SOX 9 immunohistochemistry staining (FIGS. 21C-21D); H&E staining (FIGS. 21E-21F); Safranin O staining (FIGS. 21G-21H) and Alcian blue staining (FIGS. 21I-21J) on cryosections of hUCMSCs encapsulated composite bioscaffolds at indicated time points. (Scale bar: 50 μm).

### DETAILED DESCRIPTION OF THE INVENTION

For the purposes of promoting and understanding of the principles of the invention, reference will now be made to the embodiments illustrated in the drawings and described in the following written specification. It is understood that the present invention includes any alterations and modifications to the illustrated embodiments and includes further applications of the principles of the invention as would normally occur to one skilled in the art to which the invention pertains.

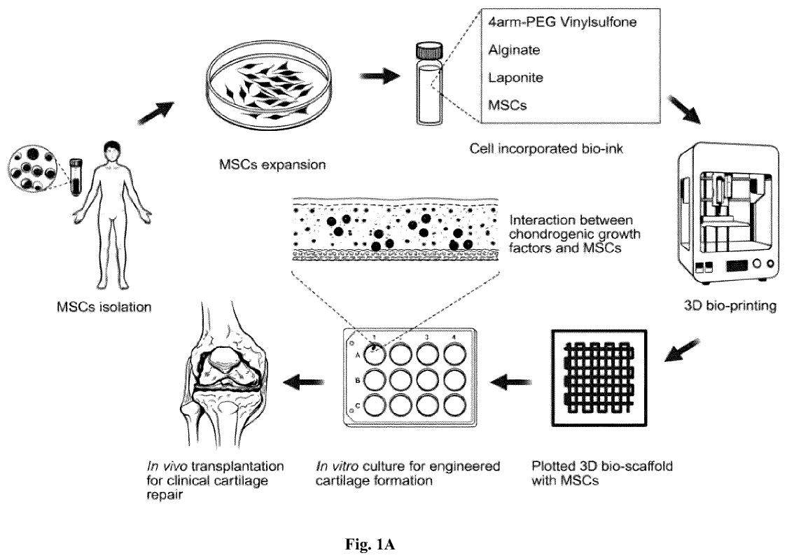

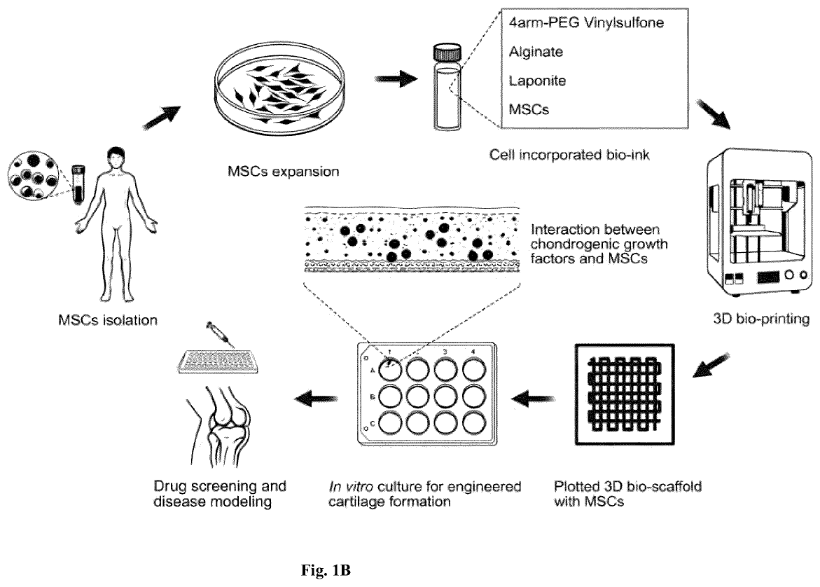

Referring to the drawings as shown in FIGS. 1 to 20, the present invention will now be described in more detail.

The present invention teaches a method for generating three-dimensional (3D) cartilage organoids through bioprinting, the method comprising: expanding mesenchymal stem cells (MSCs); preparing a composite bioink; and, bioprinting a cell-laden scaffold to generate the 3D cartilage organoids.

In accordance with a preferred embodiment of the present invention, the step of expanding the mesenchymal stem cells (MSCs) further comprising: obtaining human umbilical cord cell suspension in-vitro as a source of the MSCs; culturing the human umbilical cord cell suspension in an optimum culturing environment; and, subculturing the human umbilical cord with a new culture medium to obtain a monolayer culture of human umbilical cord-derived MSCs; and, replacing the culture medium at regular intervals until a desired cell density is achieved.

In accordance with a preferred embodiment of the present invention, the step of preparing the composite bioink further comprising: mixing sterilized water and clay, forming a water-clay mixture; dissolving a functionalized polymer and alginate powders into the water-clay-mixture forming a hydrogel solution; and, sterilizing the hydrogel solution to obtain purified composite bioink.

In accordance with a preferred embodiment of the present invention, the step of bioprinting the cell-laden scaffold to generate the 3D cartilage organoids further comprising: detaching and transferring a monolayer culture of the human umbilical cord-derived MSCs into a purified composite bioink to form a cell-laden bioink; thoroughly mixing the cell-laden bioink; removing residual bubbles by centrifuging the mixed cell-laden bioink; transferring the mixed cell-laden bioink into a 3D bioprinter with pre-determined parameters to fabricate a 3D cell-laden scaffold in the form of cubic blocks through cross-linking; and, culturing the 3D cell-laden scaffold in an inductive culture medium to generate the 3D cartilage organoids.

The present invention also discloses a three-dimensional (3D) cartilage organoid for use in disease modeling and screening, the organoid comprising: human multipotent mesenchymal stem cells (MSCs) and a composite bioink.

In accordance with a preferred embodiment of the present invention, the MSCs proliferate and differentiate into progenitor or specialized chondrogenic cells, supporting tissue development and regeneration.

In accordance with a preferred embodiment of the present invention, the composite bioink is a mixture of sterilized water, 6% w/v clay, 12% w/v functionalized polymer and 6% w/v alginate powders.

In accordance with a preferred embodiment of the present invention, the functionalized polymer includes but is not limited to polyethylene glycol (PEG) modified with vinyl sulfone (VS) groups, configured to enable thiol-based crosslinking.

In accordance with a preferred embodiment of the present invention, the clay includes but is not limited to nanoclay.

In accordance with a preferred embodiment of the present invention, the organoid includes but is not limited to a patient-specific model that replicates the anatomical structure of native cartilage to improve disease modeling and screening outcomes.

In accordance with a preferred embodiment of the present invention, the organoid has achieved a Young's modulus within a range of 137.7-182.65 kPa with a viscosity within a range of 2867.0-2928.0 Pa·s.

In accordance with a preferred embodiment of the present invention, the organoid has a breaking stress within a range of 21.2-37.8 MPa.

### Example

The invention is further described with reference to the drawings shown in FIGS. 1 to 20.

FIGS. 1A-B illustrate schematic diagrams of the preparation flow for 3D-printed cartilage organoids, their formation using bioprinting, and their applications in (A) in vivo transplantation for clinical cartilage repair, and (B) drug screening and joint disease modeling. The detailed steps for generating 3D cartilage organoids through bioprinting are further explained below.

Firstly, mesenchymal stem cells (MSCs) derived from human umbilical cords is expanded in vitro. The human umbilical cord-derived MSCs are obtained from ATCC (American Type Culture Collection) and cultured following established protocols. The frozen cells are rapidly thawed in a 37° C. water bath. The entire cell suspension from the cryovial is transferred to a 50 ml conical tube, and 5 ml of prewarmed low-glucose DMEM supplemented with 1% penicillin/streptomycin sulfate, 1% L-glutamine, and 10% fetal bovine serum is added. After an additional 5 mL of complete medium is introduced, the entire cell suspension is transferred to a culture plate and is incubated at 37° C. in a humidified atmosphere containing 5% CO2. When the cells undergo their first subculture, the medium is replaced with complete MesenPRO RS medium. The adherent cells are maintained with medium changes every two days until they reach confluence.

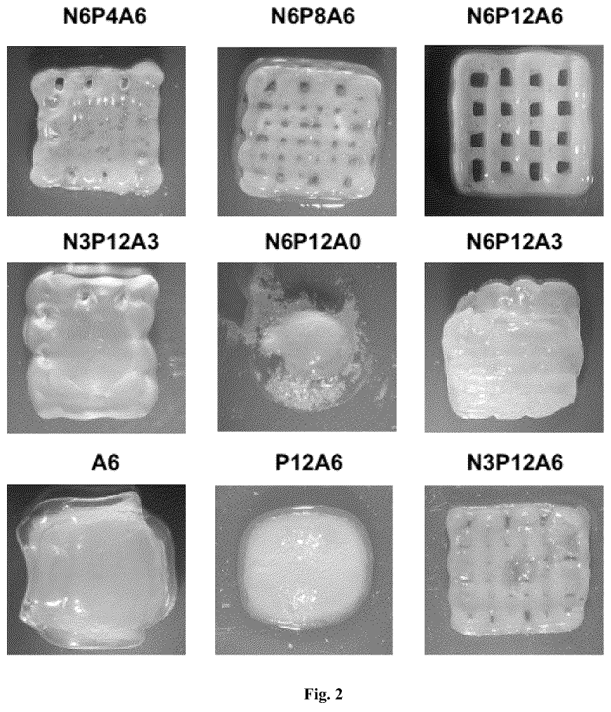

Then, to develop a composite bioink with high printability, various formulations with different compositions and concentrations are tested for their performance in 3D plotting. A printability analysis and bioink optimization are conducted. This is shown in FIG. 2 where a printability evaluation of different bioinks under various parameters is conducted. The bioink containing nanoclay (NC), polyethylene glycol (PEG), and alginate (ALG) exhibits high printing accuracy after plotting. Table 1 presents nomenclature of the tested bioinks (all percentages by weight).

TABLE 1

| Formula | Nanoclay | 4 arm PEG-VS | Alginate |
|---|---|---|---|
| N6P4A6 | 6 w/v % | 4 w/v % | 6 w/v % |
| N6P8A6 | 6 w/v % | 8 w/v % | 6 w/v % |
| N6P12A6 | 6 w/v % | 12 w/v % | 6 w/v % |
| N3P12A3 | 3 w/v % | 12 w/v % | 3 w/v % |
| N6P12A0 | 6 w/v % | 12 w/v % | 0 w/v % |
| N6-12-3 | 6 w/v % | 12 w/v % | 3 w/v % |
| A6 | 0 w/v % | 0 w/v % | 6 w/v % |
| P12A6 | 0 w/v % | 12 w/v % | 6 w/v % |
| N3P12A6 | 3 w/v % | 12 w/v % | 6 w/v % |

The results reveal that pure PEG and PEG-NC mixtures fail to form stable 3D structures, as the printed strands collapse immediately, indicating poor printability. In contrast, a paste combining PEG and ALG is printable, but its strand morphology is unstable; printed scaffolds retain their shape only briefly before crosslinking. Although this issue may be improved by increasing ALG content to further enhance the material's viscosity and maintain print quality, it requires higher pneumatic pressure for extrusion, which harms cell viability. However, a composite bioink combining NC, PEG and ALG (NC-PEG-ALG) demonstrates superior plotting performance and enables extrusion at a moderate pressure while maintaining excellent shape fidelity in the plotted structures. The term “composite” refers to “NC-PEG-ALG” and is used interchangeably herein.

NC-PEG-ALG bioink is prepared by blending 6% w/v of NC with 12% w/v 4-arm PEG-VS solution and 6% w/v of ALG. After sterilizing the NC, PEG, and ALG powders under UV irradiation for 2 hours, NC powder is gradually added to sterile deionized water (DW) in a 3 ml syringe. The syringe containing the clay solution is then connected to a second syringe using a syringe connector, and the mixture of water and clay is thoroughly blended by repeatedly pushing the syringe piston. ALG and PEG-VS powders are subsequently added to the clay solution, allowing them to dissolve through continuous mixing with the syringe and sonication for 1 hour. The final hydrogel solution is further sterilized by UV irradiation for 30 minutes. All subsequent results related to NC-PEG-ALG scaffolds are fabricated from this material. For alginate bioink (ALG), sterile alginate powder (6% w/v) is dissolved in DW under a Class II laminar flow cabinet. The solution is mixed in syringe and treated with sonication for 1 hour and stored at 4° C. overnight.

The concentration used for material fabrication is determined and validated through printability and rheology tests. The results indicate a correlation between the material's concentration and its printability, rheological, and mechanical properties. Therefore, the concentration specified in the patent should be precisely followed during the material synthesis process. However, slight variations in concentration are inevitable due to systematic errors, such as the limited decimal precision of the weighing balance. Despite this, these variations should be minimized. To ensure the fabricated biomaterial achieves optimal printability, shape fidelity, and mechanical properties, the acceptable concentration range is 6±0.1% w/v clay, 12±0.1% w/v functionalized polymer, and 6±0.1% w/v alginate.

Further, cell-laden scaffolds are bioprinted using the NC-PEG-ALG bioink to generate the 3D cartilage organoids. Confluent monolayer cultures of human umbilical cord-derived MSCs are detached using trypsin-EDTA and then are suspended in serum-free high-glucose DMEM. The cell suspension is then transferred into a 3 ml syringe containing 1 mL of composite bioink, and the mixture is thoroughly blended. The cell-laden bioink is transferred to a 10 mL 3D printing cartridge and is centrifuged at 2000 g for 5 minutes to remove any residual bubbles. A printing tip with an inner diameter of 420 μm is attached to the cartridge, which is then mounted onto the 3D extrusion printer. After calibrating the cartridge position, the printing pressure is set to ensure the continuous extrusion of the cell-laden composite bioink. Cubic-shaped blocks are printed and crosslinked using CaCl₂) and 2,2-(ethylenedioxy) diethanethiol solution. Following crosslinking, the printed 3D cell-laden block is cultured in chondrogenic media to promote chondrogenic differentiation of the encapsulated MSCs and cartilage organoid formation.

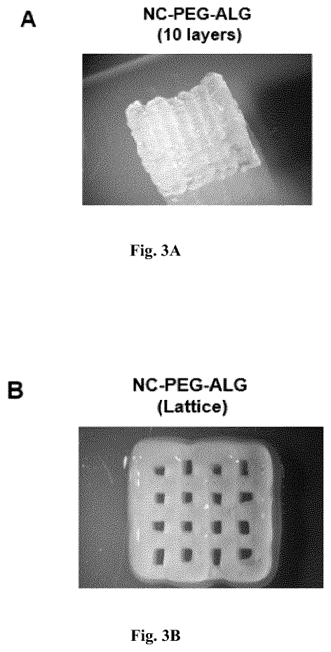

To evaluate print accuracy, the NC-PEG-ALG bioink is extruded in air using a 0.4 mm inner-diameter printing tip, without the addition of stabilizing agents. After crosslinking with CaCl₂) and 2,2-(ethylenedioxy) diethanethiol respectively, the printed constructs exhibit high precision in clinically relevant dimensions (10×10 mm²) and shapes. FIGS. 3A-B which illustrate cell-laden scaffolds with various shapes and dimensions. FIG. 3A shows a top view of a 10-layer cube that retains a consistent shape as its height increases, while FIG. 3B presents a top view of well-defined printed strands in a lattice structure.

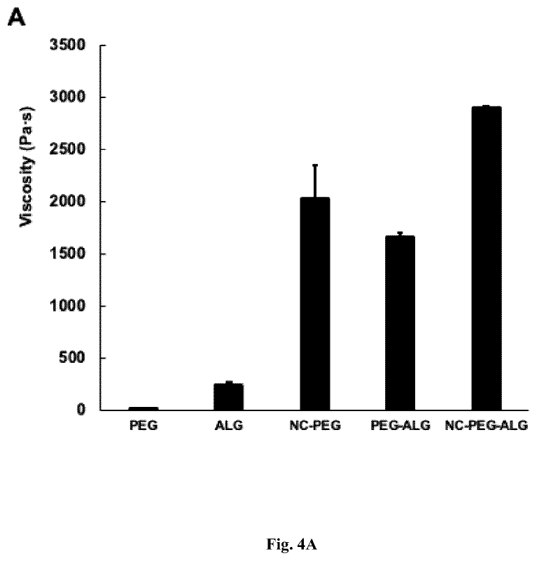

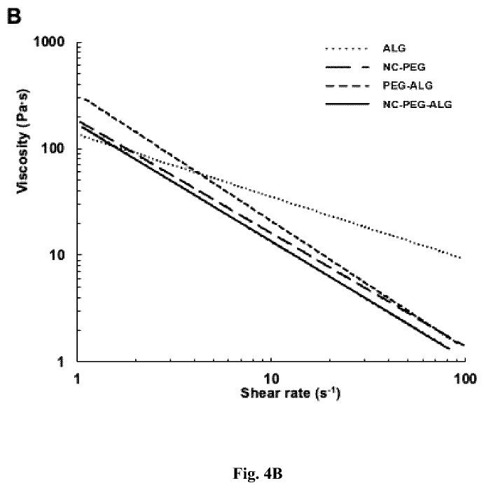

Further, the rheological properties of various bioinks are evaluated using a plate rheometer with a plate-plate distance of 0.2 mm under constant and increasing shear rates. Viscosity measurements shows that pure PEG exhibited a low viscosity of 9.1±0.9 Pa·s at a shear rate of 0.1 s⁻¹, correlating with its poor printability. In contrast, ALG displays a significantly higher viscosity of 246.9±17.1 Pa·s. The combination of ALG and PEG (PEG-ALG) further increases the viscosity to 1666.3±43.4 Pa·s. When NC is added, a substantial viscosity increase is observed. With 6% NC addition (NC-PEG-ALG), the viscosity rises to 2904.3±11.8 Pa·s, demonstrating the ability of the printed strands to maintain a stable 3D structure. In comparison, the NC-PEG bioink without ALG had a lower viscosity of 2035.2±307.0 Pa·s. All compositions exhibit shear-thinning behavior under increasing shear rates ranging from 1 to 100 s⁻¹, facilitating extrusion through nozzles, as shown in FIGS. 4A-B. Said figures present the rheological properties of different bioinks NC, PEG and ALG at room temperature. FIG. 4A illustrates viscosity at a constant shear rate of 0.1 s−1. It is seen that the low viscosity of PEG is increased by the addition of ALG and further strongly increased by blending with NC. Whilst FIG. 4B illustrates a shear thinning effect of bioinks at increasing shear rates.

The bioinks exhibit viscoelastic behavior and are tested within the linear viscoelastic region. The storage modulus (G′) represents the elastic, gel-like component, while the loss modulus (G″) corresponds to the viscous, non-gelled component. Across a wide range of angular velocities, all bioinks demonstrate a higher storage modulus than loss modulus, indicating predominant elastic behavior. For ALG, PEG-ALG, and NC-PEG bioinks, the storage modulus remains higher up to an angular velocity of 100 Hz. Beyond this crossover point, the elastic properties are diminished, and the bioinks behave as viscous fluids, as indicated by the dominance of the loss modulus. In contrast, the NC-PEG-ALG bioink maintains a higher storage modulus than loss modulus throughout the tested range, signifying a stable and robust internal network. Similarly, amplitude sweep, and frequency sweep tests are conducted to evaluate the viscoelastic behavior of crosslinked hydrogels. All compositions consistently show a higher storage modulus than loss modulus across a broad range of strain and angular velocities. The NC-PEG-ALG composition exhibits particularly strong internal network stability, as evidenced by its stable storage modulus under increasing strain and a trend of increasing storage modulus with rising frequency.

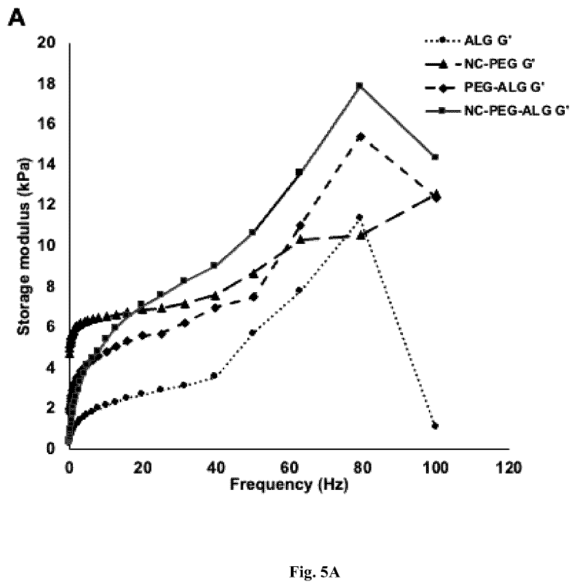

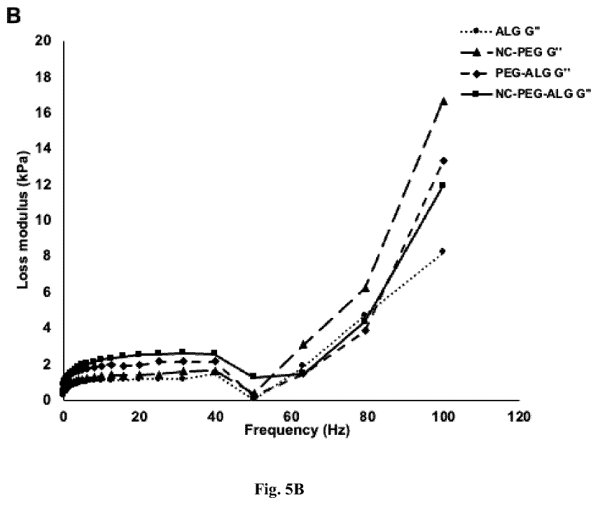

FIGS. 5A-D illustrate viscoelastic behavior of different bioinks and crosslinked hydrogel consisting of NC, PEG and ALG at room temperature. FIGS. 5A and B illustrate frequency sweep measurements of different bioinks. It displays that all of the bioinks show a storage modulus (G′) higher than the loss modulus (G″). For NC-PEG-ALG composite bioink, a lower loss modulus is observed at higher frequency. FIG. 5C illustrates amplitude sweep measurements of different printed hydrogel ranging from 0% to 10% strain, and FIG. 5D illustrates frequency sweep measurements of different printed hydrogel ranging from 0.1 Hz to 2 Hz.

Energy-dispersive X-ray spectrometry (EDX), a technique used for surface analysis and element characterization of organic and inorganic samples, is employed to assess the presence of NC and its integration within the printed structures. Cell-free NC-PEG-ALG and ALG bioinks are printed in 3D according to established conditions. After printing and crosslinking, the scaffolds are dehydrated by freeze-drying for characterization. During the EDX analysis, conducted at a voltage of 10 kV, the sample is bombarded with a high-voltage electron beam. The emitted back-scattered and secondary electrons are detected to construct a point-by-point image of the specimen. Additionally, the interaction between the electron beam and the sample causes the emission of X-ray radiation. By correlating the X-ray wavelength and intensity with the position of the electron beam, an elemental map of the specimen is generated. Additionally, the quantification and ratio of elements are determined based on the emitted energy intensity.

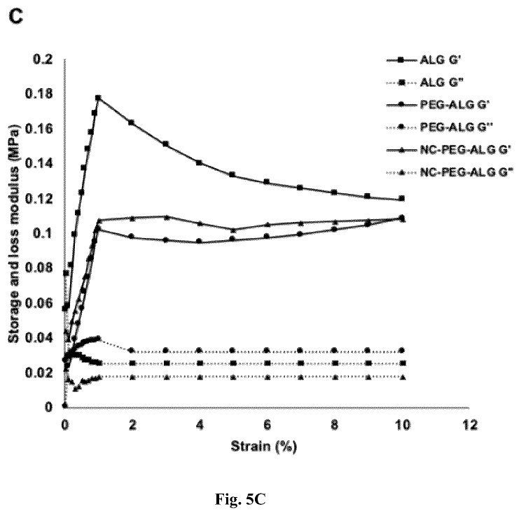

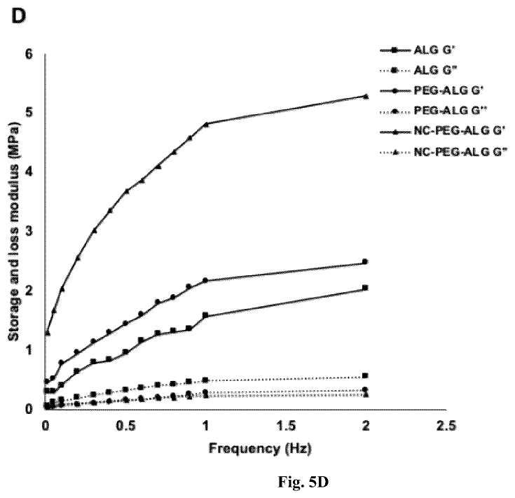

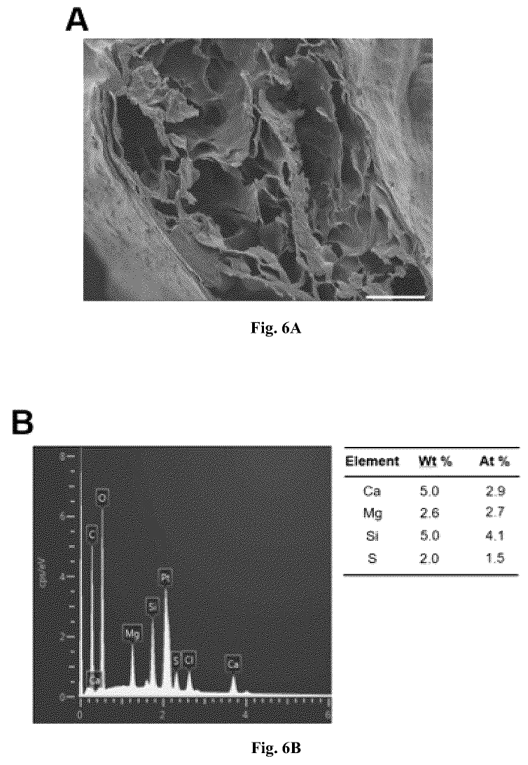

As shown in FIGS. 6A-C, a small amount of sodium is detected, originating from the ALG and NC crystal structure. Calcium ions are also observed, attributed to crosslinking with the CaCl₂) solution. Elemental mapping reveals the distribution of silicon and 4 magnesium, indicating the uniform dispersion of NC across the strand's cross-section. Furthermore, sulfur from PEG-VS and silicon are found to be homogeneously distributed, as evidenced by overlapping detection areas, confirming the integration of PEG-VS and NC within the structure. FIGS. 6A-C illustrate EDX and EDX mapping of a cross section of a printed NC-PEG-ALG bioscaffold strand, wherein (A) and (B) illustrates EDX element analysis revealed characteristic peaks of NC-PEG-ALG; (C) illustrates mapping of Si and Mg, and its merge showed distribution of NC in the strand of scaffold, while element mapping of S and Si and its merge revealed integration of PEG-VS and NC.

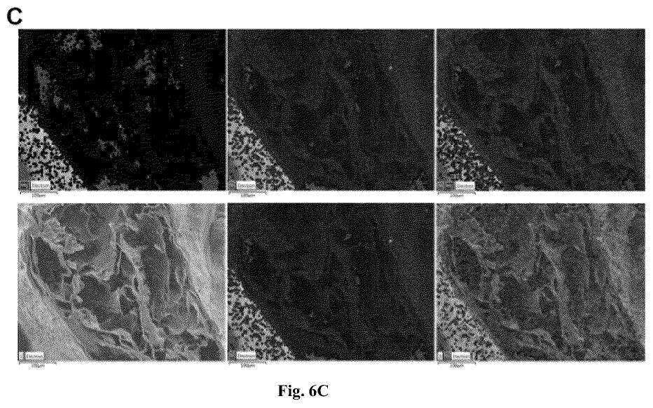

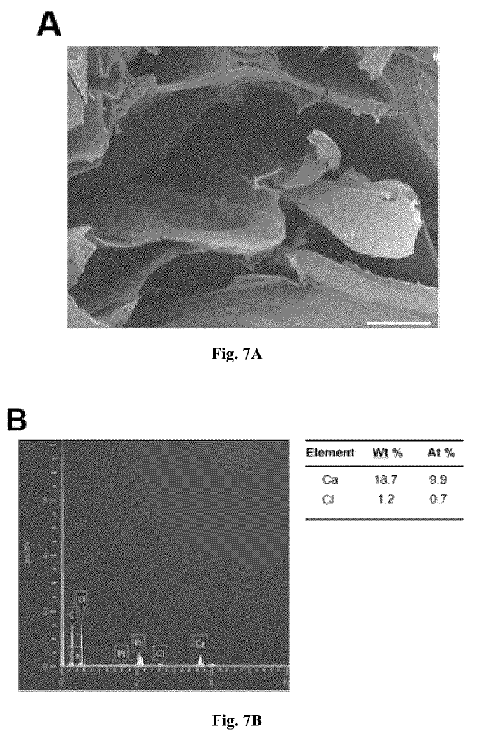

Meanwhile, for ALG printed scaffold, the signal of calcium is captured due to the usage of CaCl₂) as crosslinker and shown in FIGS. 7A-C. Sodium arising from ALG is also scanned. FIGS. 7A-C illustrate EDX and EDX mapping of a cross section of a printed ALG bioscaffold strand. FIGS. 7A and B illustrate characteristic peaks of ALG, and FIG. 7C illustrates an element mapping of Ca showed the crosslinked strand of the bioscaffold. The scale bars for FIGS. 7A and Care 100 μm.

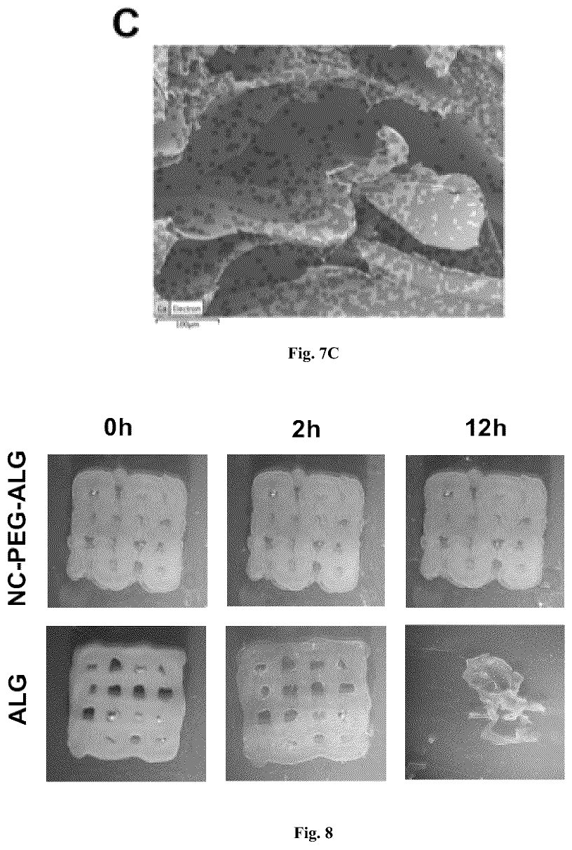

Compared to traditional ALG hydrogels formed through ionic crosslinking, the NC-PEG-ALG hydrogel achieves double crosslinking by incorporating both physical crosslinking via calcium ions and chemical crosslinking through a thiol group-initiated Michael-type addition reaction. To verify the formation of this double crosslinking, ALG hydrogel and NC-PEG-ALG scaffolds are immersed in 100 mM citric acid, which acts as a calcium chelator by effectively removing calcium ions from the hydrogel. Even after the elimination of calcium ions, the double-crosslinked NC-PEG-ALG scaffold retains its lattice structure, confirming the presence of double crosslinking. While the calcium-ion mediates physical crosslinking is reversible, the covalent bonds formed through chemical crosslinking ensure the gel's structural integrity. In contrast, the ionically crosslinked ALG hydrogel partially dissolves in the citric acid solution after 12 hours of incubation, due to the loss of calcium ions and the subsequent collapse of the hydrogel network. FIG. 8 illustrates undissolved bioscaffolds treated with 100 mM citric acid. It demonstrates that the stable double crosslinking of NC-PEG-ALG is achieved.

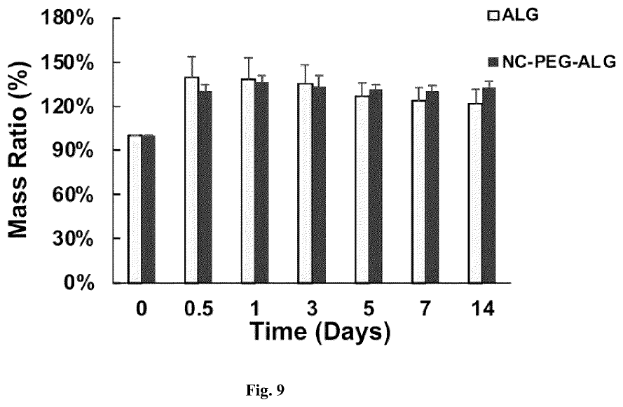

Further, the in vitro stability of the materials is also assessed. Firstly, the mass swelling ratio of the ALG and NC-PEG-ALG scaffolds, an important parameter indicating the materials' swelling capacity, is measured by weighing the dry scaffolds and then tracking the wet weight of incubated scaffolds at specific time intervals. After a 14-day incubation, the NC-PEG-ALG scaffold's mass swelling ratio reaches 132.9%. FIG. 9 shows mass swelling ratio characterization of ALG hydrogel and NC-PEG-ALG scaffold.

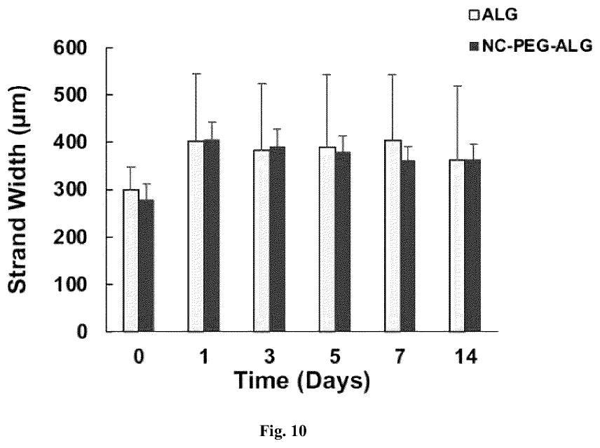

To further evaluate the shape retention capacity of the materials, strand width analysis is conducted. Following crosslinking in a 100 mM CaCl₂) solution, both NC-PEG-ALG and pure alginate bioscaffolds (ALG) are cultured in PBS under physiological conditions for 14 days. At set time points, scaffold images are taken, and strand diameters are measured. During 24 hours of PBS incubation, the strand diameter of the NC-PEG-ALG scaffolds increases to 405 μm±37 μm, indicating strand swelling during crosslinking. In the following days, strand diameter remains relatively stable, with a diameter of 392 μm±37 μm on day 3. After 7 days, the strand diameter has decrease slightly to 361 μm±46 μm, suggesting that the strands reached a stable state with minimal swelling by day 14. Overall, the NC PEG-ALG scaffolds swell by approximately 33.3% after plotting and subsequent incubation in PBS at physiological conditions (37° C.) for 14 days. In comparison, the pure ALG scaffold exhibits a strand diameter of 362 μm±157 μm over the 14-day culture, with significant variation. The high variance in strand width indicates that the ALG scaffolds swell unevenly and fail to maintain their original shape throughout the incubation period. FIG. 10 shows strand width change of ALG scaffold and NC-PEG-ALG scaffold.

Additionally, while micro-fractures appear on the ALG scaffolds, no defects are observed on the NC-PEG-ALG bioscaffolds, indicating that the NC-PEG-ALG biomaterial has a promising in vitro stability. FIG. 11 illustrates a morphology of generated strands from NC-PEG-ALG and ALG hydrogel incubated in PBS for 7 days.

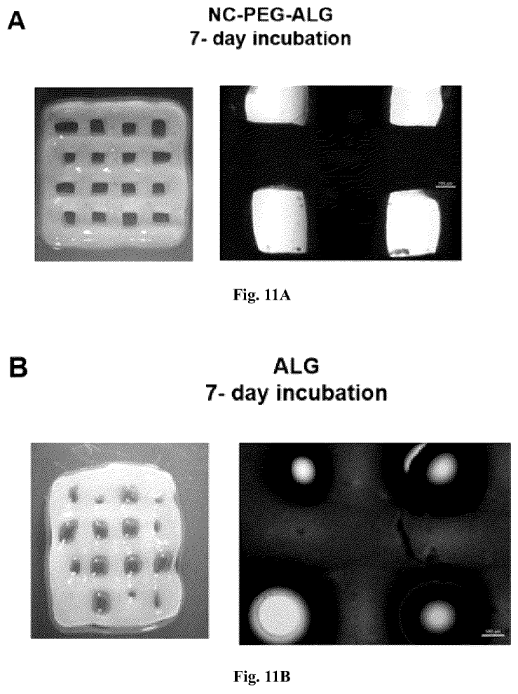

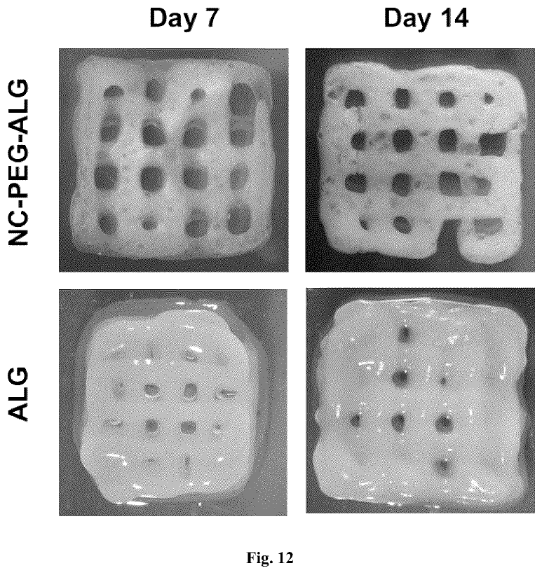

Besides, the durability of cell-laden 3D bioprinted cartilage organoids are assessed using chondrocytes isolated from newborn mice. The cells are cultured in αMEM medium supplemented with 10% FBS, 1% Pen/Strep, and 1% L-Glutamine. After harvesting, 5×10⁶ cells are suspended in 20 μL of culture medium and mixed into 1 g of NC-PEG-ALG and ALG bioinks, respectively. The cell-laden cartilage organoids are 3D printed following established parameters and incubated in αMEM culture medium for 14 days. Microscopic images of the constructs are captured at specific time intervals using a dissection microscope. The results reveal that throughout the incubation period, NC-PEG-ALG scaffolds preserve their intended three-dimensional architecture, showcasing excellent structural durability. In contrast, the ALG-based cell-laden constructs, like their cell-free counterparts, exhibit partial swelling, highlighting their limited durability under in vitro conditions. FIG. 12 illustrates cell encapsulated ALG scaffold and composite scaffold incubated in culture medium at 37° C. The composite scaffolds maintain complete lattice-structure at day 14, indicating their in vitro durability.

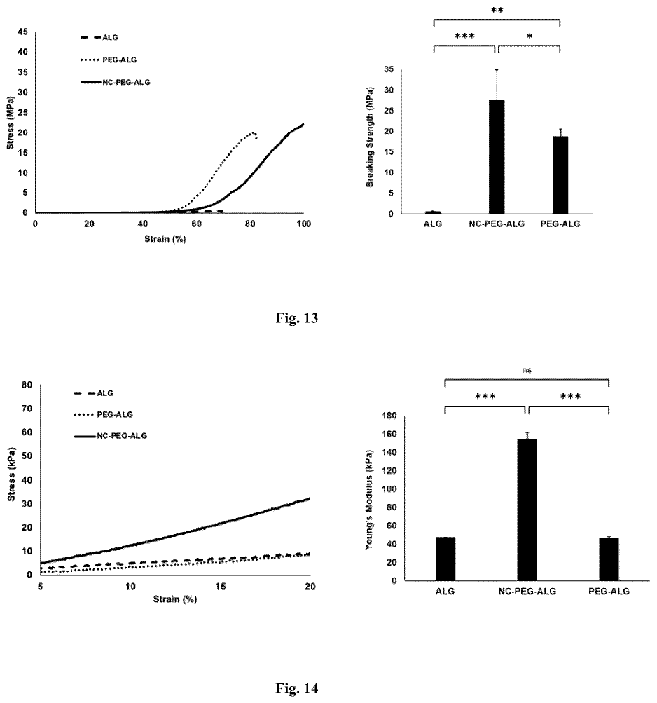

Additionally, mechanical tests are performed to evaluate whether the mechanical properties of the printed NC-PEG-ALG bioscaffold align with those of native cartilage. First, the compressive breaking stress of different materials is measured by compressing the prepared samples until structural collapse. Results show that the nanoclay-incorporated NC-PEG-ALG bioscaffold achieves a breaking stress of 27 MPa, which is significantly higher than that of conventional ALG hydrogels formed by physical crosslinking or the PEG-alginate mixture. FIG. 13 shows breaking compressive stress comparison among 3D bioprinted ALG, PEG-ALG, and the NC-PEG-ALG bioscaffolds.

This underscores the crucial role of NC in improving scaffold strength. Notably, the breaking stress of this hydrogel surpasses the typical stress range experienced by human cartilage (5-10 MPa). Additionally, a stiffness test is conducted on the NC-PEG-ALG bioscaffolds. Through stress-strain analysis in the linear elastic region, the Young's modulus of the printed bioscaffolds is determined. The NC-PEG-ALG scaffold exhibits a Young's modulus of 154 kPa, significantly higher than that of pure ALG and PEG-ALG scaffolds and closely matching the Young's modulus of human cartilage ECM, ranging from 130 kPa to 573 kPa. FIG. 14 illustrates Young's modulus comparison among 3D bioprinted ALG, PEG-ALG, and the NC-PEG ALG bioscaffolds.

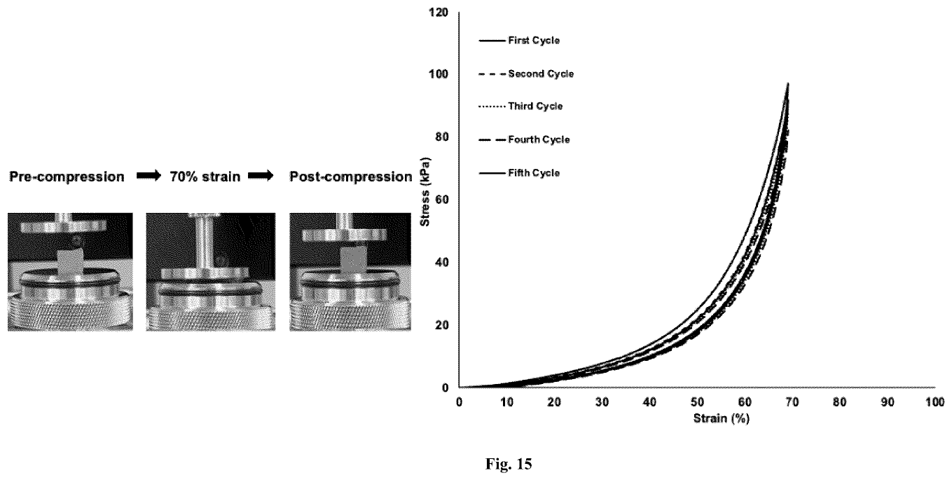

This high mechanical strength and biomimetic stiffness make the NC-PEG-ALG scaffold highly suitable for cartilage repair. The NC-PEG-ALG hydrogel structures also demonstrate notable deformability and self-recovery. Printed hydrogel cubes are repeatedly compressed to 30% of their original height, then is allowed to return to their initial shape. The recorded stress-strain curves are highly consistent across cycles, showing that after five deformation cycles, the cubes exhibit minimal permanent deformation and quickly regained their original height upon unloading. FIG. 15 illustrates self-recovery profile of the NC-PEG-ALG bioscaffold.

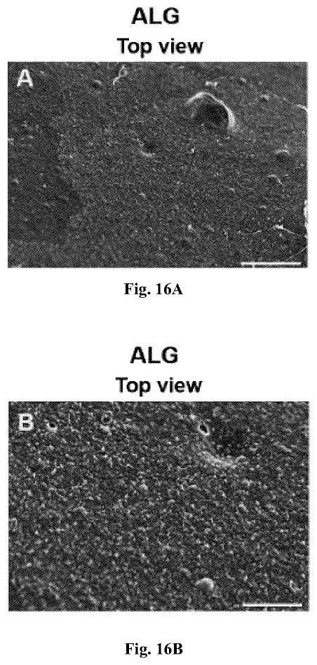

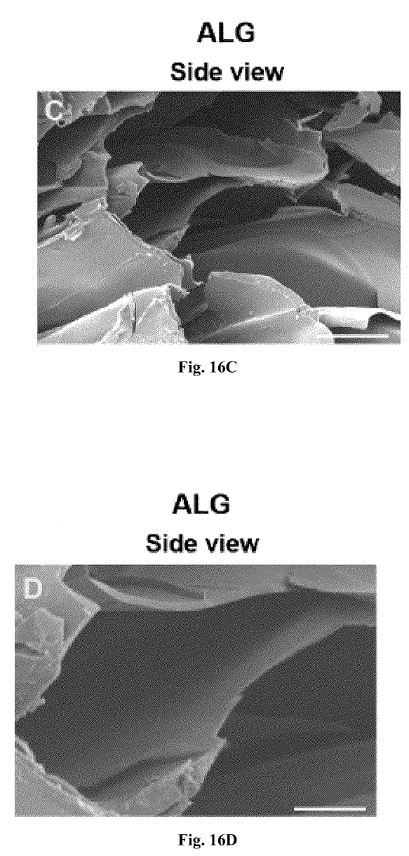

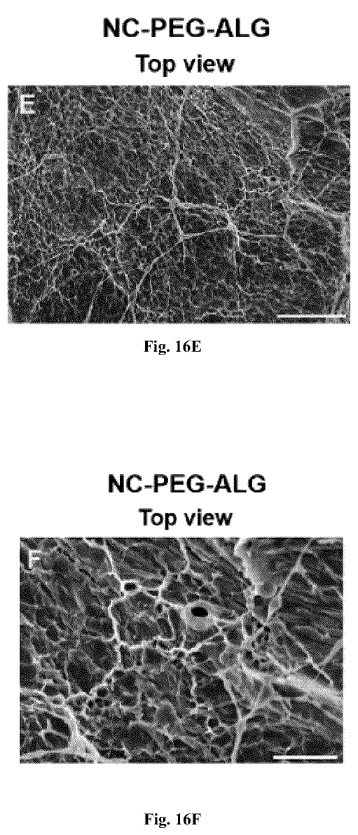

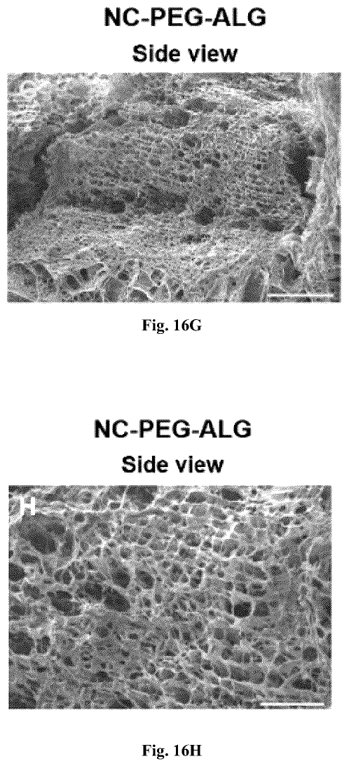

The microstructure of the printed bioscaffolds is investigated using scanning electron microscope (SEM) analysis on crosslinked NC-PEG-ALG hydrogels. Freeze-dried samples of both ALG hydrogel and NC-PEG-ALG hydrogel are gold sputter-coated using a high-vacuum ion sputtering coater prior to observation. The structure and surface morphology of the crosslinked NC-PEG-ALG and ALG bioscaffolds are then examined. SEM images reveal a nanoporous structure with fibrous aggregates in the NC-PEG-ALG hydrogels as shown in FIG. 16E, in contrast to the relatively uniform appearance of freeze-dried ALG gels at the same submicron scale shown in FIG. 16A. This finding indicates that the crosslinking process creates a more rigid network in the NC-PEG-ALG hydrogel, contributing to its enhanced mechanical properties. Furthermore, the smaller pore diameter in the NC-PEG-ALG hydrogel provides a more 3D adhesion configuration for cells. This limits the surface area available for monolayer proliferation, encouraging cells to aggregate in 3D. Such cell aggregation is a critical factor for the phenotypic expression of chondrocytes. FIGS. 16A-H illustrate a scanning electron microscope characterization of the NC-PEG-ALG bioscaffold and ALG bioscaffold. FIGS. 16A-D illustrate top view and side view of the dehydrated alginate hydrogel with homogeneous appearance and low roughness, while FIGS. 16E-H illustrate top view and side view of the freeze-dried composite hydrogel with nanoscale porous structure. The scale bars used in the figures are 20 μm for FIGS. 16(A) and (E), 100 μm for FIGS. 16(C) and (G), 700 nm for FIGS. 16(B) and (F), and 30 μm for FIGS. 16(D) and (H).

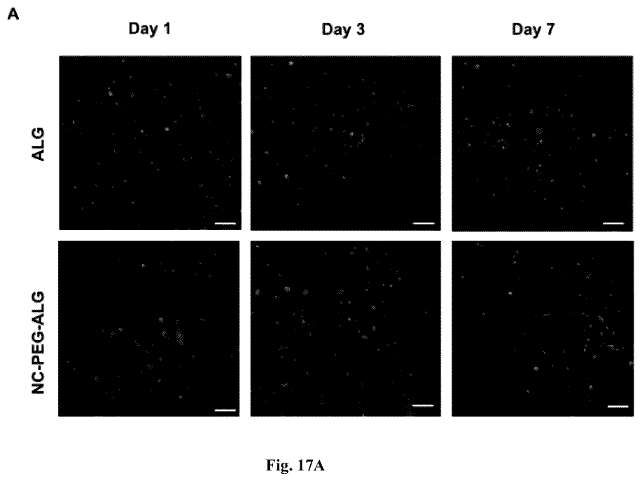

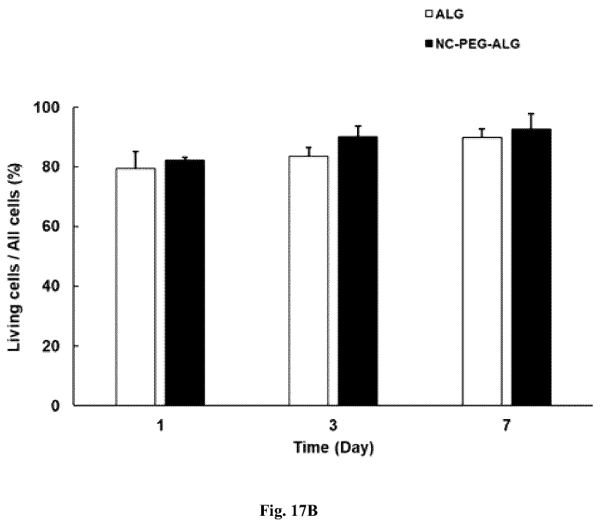

Maintaining high cell viability is crucial for the effectiveness of hydrogel bioinks. To assess the biocompatibility of the fabricated NC-PEG-ALG material, primary chondrocytes isolated from the ribs of C57BL/6 newborn mice are encapsulated in the hydrogel at a density of 5×10⁶ viable cells/mL. The viability of cells within the PEG-based organoid constructs is evaluated at days 1, 3, and 7 post bioprinting. At each time point, the samples are washed with PBS, transferred to a 12-well plate, and stained with 1 μM calcein-AM (excitation/emission wavelengths of 488/515 nm) for 30 minutes at 37° C., followed by 0.75 M propidium iodide (excitation/emission wavelengths of 561/625 nm) for 20 minutes at 37° C. The samples are then washed three times with PBS and imaged using an inverted fluorescence microscope. Fluorescent imaging of the cell-laden NC-PEG-ALG scaffold, stained with Calcein-AM and Ethidium homodimer-1, shows high cell viability for up to 7 days of culture following bioprinting. This is shown in FIG. 17A. Quantitative analysis reveals that the NC-PEG-ALG hydrogel supported over 80% cell viability at all time points, confirming that the NC-PEG-ALG organoid exhibits excellent biocompatibility and supports sustained high cell viability as shown in FIG. 17B. FIGS. 17A-B illustrate cell viability analysis of the 3D bioprinted cartilage organoids, where FIG. 17A illustrates LIVE/DEAD staining for chondrocytes encapsulated in ALG cartilage organoids and the NC-PEG-ALG cartilage organoids, and FIG. 17B illustrates viability of encapsulated cells in ALG cartilage organoids and the NC-PEG-ALG cartilage organoids cultured up to 7 days. The scale bars used in the figures are 100 μm.

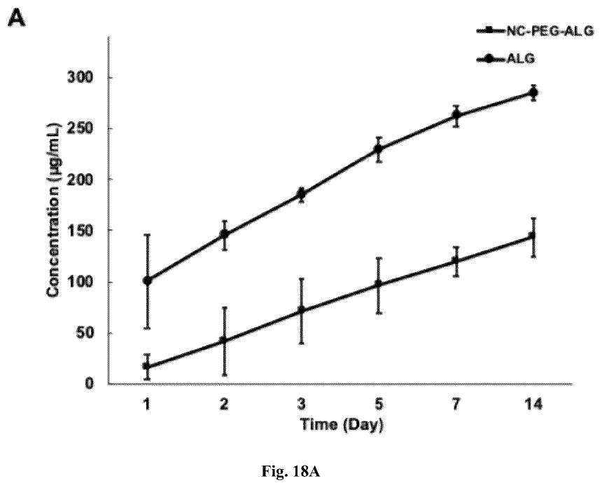

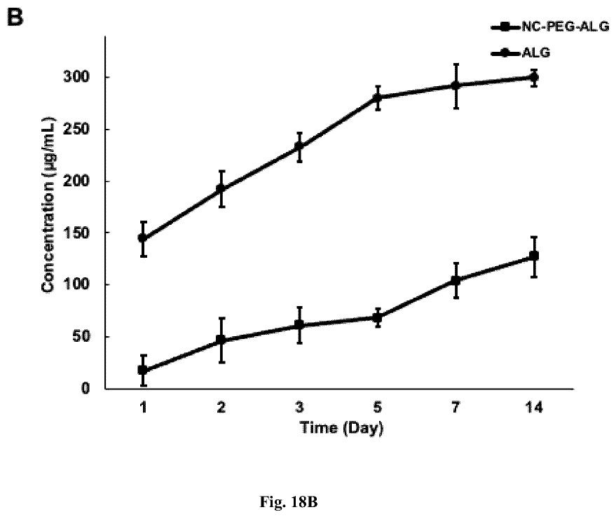

To determine whether the incorporation of nanoclay enhances protein absorption and retention, bovine serum albumin (BSA) and lysozyme are used as model proteins. These proteins are solubilized in deionized water at a concentration of 1.5 mg/mL. NC-PEG-ALG bioscaffolds consisting of NC, PEG and ALG, as well as pure ALG gels of the same volume, are soaked in the protein solutions for 1, 2, 3, 5, and 7 days. At each point, the supernatant is collected to analyze the protein absorption profile via Bradford protein quantification assay. For the release profile, concentrated protein solutions (4.5 mg/mL) are mixed with the bioinks before printing. The protein-laden bioscaffolds are then incubated in PBS for 1, 2, 3, 5, 7, and 14 days, and the corresponding supernatants are collected to measure the amount of protein released from the materials. According to the results of both groups' protein absorption profiles, clay incorporated NC-PEG-ALG material shows an increased absorption of BSA for all the time points. While the total amount of lysozyme absorbed by each gel is similar over a longer period, the cumulative lysozyme concentration in the NC-PEG ALG bioscaffolds is higher than in the pure ALG gel after saturation on day 1. This suggests that although the equilibrium concentrations of both materials are similar, the addition of NC facilitates faster absorption. FIGS. 18A-B illustrate release profile of model proteins from the NC-PEG-ALG bioscaffolds and ALG bioscaffolds, wherein FIG. 18A shows the in vitro cumulative release curve of BSA from NC-PEG-ALG hydrogel and ALG hydrogel, while FIG. 18B presents the cumulative release curve of lysozyme from the same hydrogels.

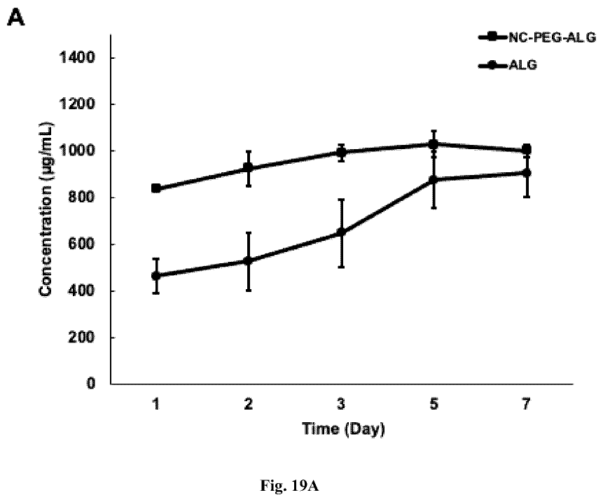

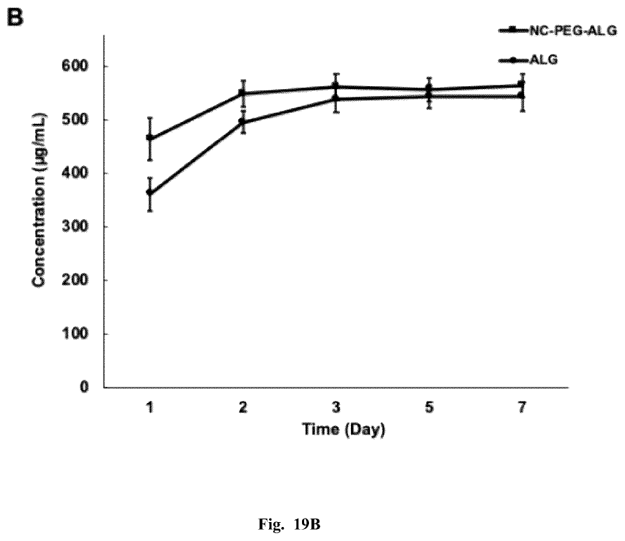

Similarly, the release profiles demonstrates that lower amounts of BSA and lysozyme are released from the NC-PEG-ALG bioscaffold containing clay at all time points, indicating more effective retention of biomolecules within the gel due to the presence of nanoclay, FIG. 19A illustrates the absorption of BSA in NC-PEG-ALG bioscaffolds and ALG bioscaffolds, while FIG. 19B presents the absorption of lysozyme in the same bioscaffolds.

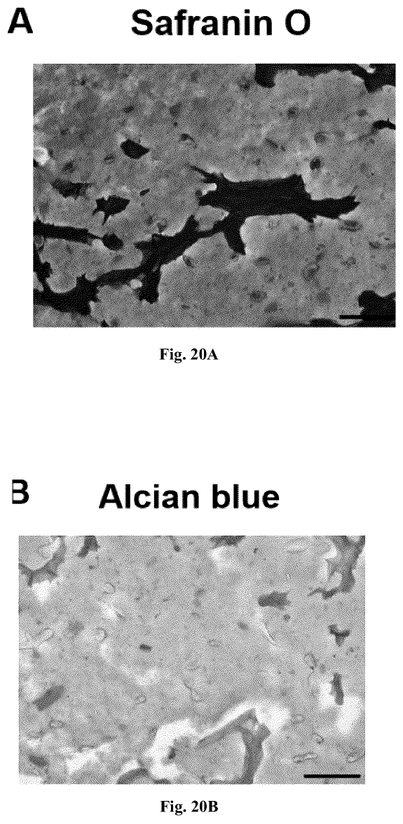

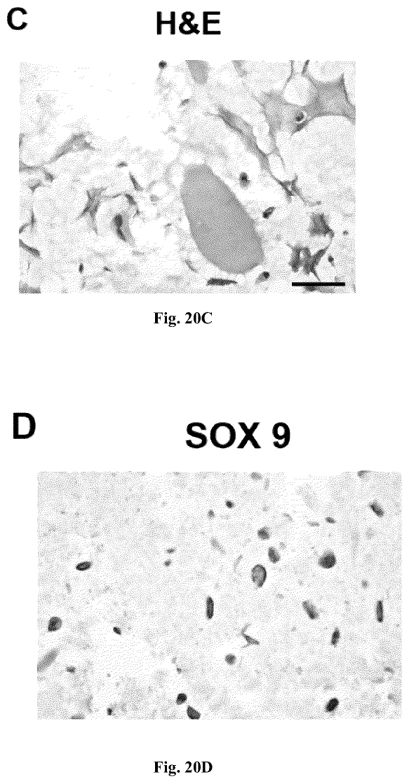

To evaluate whether the NC-PEG-ALG material able to provide a supportive microenvironment for chondrogenic cells, facilitating their differentiation, primary murine chondrocytes are encapsulated to generate the 3D bioprinted cartilage organoids. After a 14-day incubation, the cartilage organoids are fixed with 4% paraformaldehyde (PFA), embedded in OCT compound, and sectioned into thin slices (10 μm) for analysis. In chondrocyte-laden scaffolds, H&E staining reveals that the encapsulated cells maintained a spherical shape, contrasting with the spindle-like morphology typical of 2D cultures, indicating successful preservation of their phenotype as shown in FIG. 20C.

Histochemical analysis is conducted on various ECM components, with Alcian blue staining highlighting glycosaminoglycans, Safranin O staining indicating proteoglycans, and immunohistochemistry for Col II and SOX9 confirming the cartilage phenotype. The staining results clearly show the expression of chondrocyte-specific ECM and chondrogenic markers, demonstrating that the encapsulated chondrocytes retain their characteristic phenotypes and undergo differentiation, which is essential for cartilage formation and maintenance as shown in FIGS. 20A, 20B, 20D, and 20E. These findings indicate that the cell phenotype is well preserved within the 3D bioprinted cartilage organoids and suggest that the NC-PEG-ALG scaffolds support the growth of chondrogenic cells, highlighting their potential for cartilage tissue engineering and potential therapeutic applications. FIGS. 20A-E illustrates histological staining and immunostaining of chondrocytes embedded NC-PEG-ALG bioscaffolds following culture over 2 weeks. Shown are representative images of Safranin O staining (A); Alcian blue staining (B); H&E staining (C); Anti-SOX9 (D) and Anti-Col II immunohistochemistry staining (E) on cryosections of chondrocytes encapsulated NC-PEG-ALG bioscaffold at day 14. The scale bars used in the figures are 50 μm.

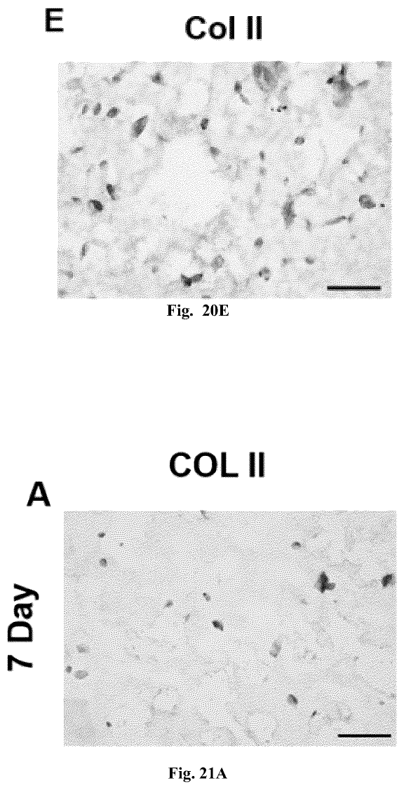

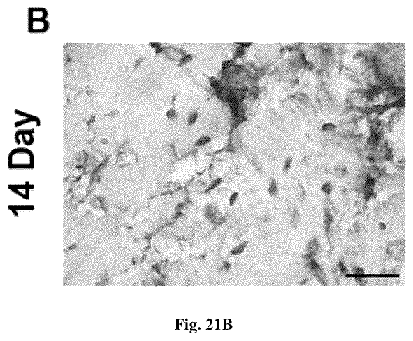

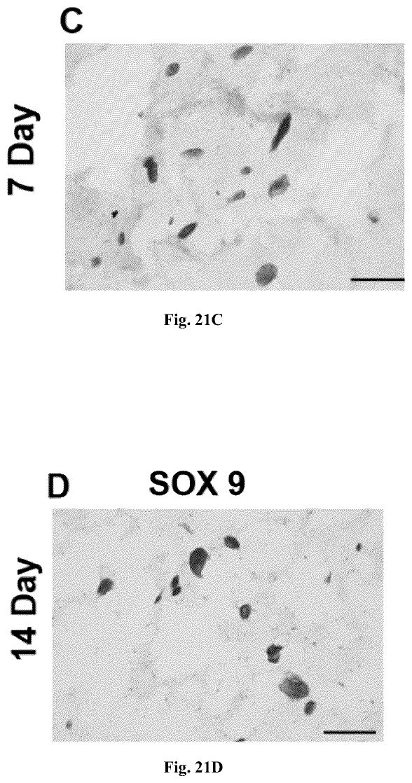

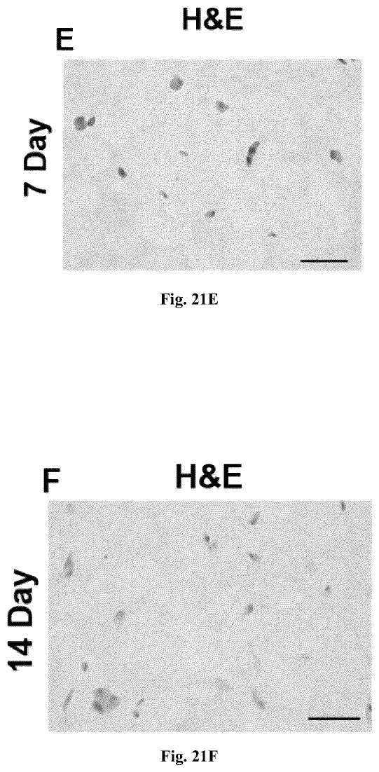

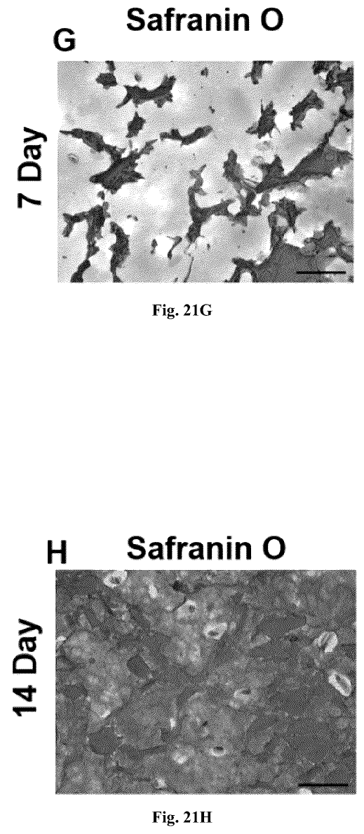

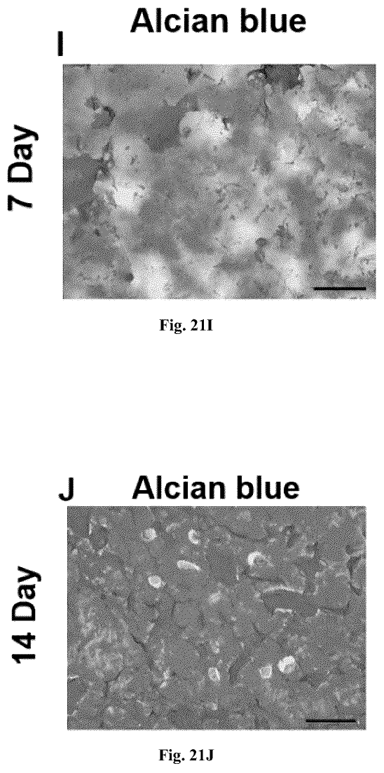

To evaluate whether the NC-PEG-ALG composite biomaterial promotes chondrogenesis from mesenchymal stem cells (MSCs), hUCMSCs were encapsulated within the NC-PEG-ALG scaffold and incubated in chondrogenic medium (high-glucose DMEM supplemented with 1% PS, 10 mM sodium pyruvate, 0.1 μM dexamethasone, 40 μg/mL L-proline, 50 μg/mL ITS premix, and 10 ng/mL TGF-β3) for 7 day and 14 days. Immunohistochemistry staining was then performed to detect the expression of chondrogenic markers and chondrocyte-specific extracellular matrix (ECM) components. As shown in FIG. 21A-D, a significant expression of SOX9 and COL II was observed after two weeks of chondrogenic induction, suggesting that the encapsulated MSCs effectively underwent chondrogenic differentiation within the hydrogel matrix. Histological analysis further confirmed MSC chondrogenesis in the NC-PEG-ALG scaffold. Hematoxylin and eosin (H&E) staining revealed the morphology of the encapsulated cells (FIGS. 21E-F), clearly demonstrating that the chondrocytes maintained a spherical shape, rather than the typical spindle-like morphology observed in 2D cell culture. Various ECM components were assessed using histochemical staining methods, including Alcian blue staining for glycosaminoglycans and Safranin O staining for proteoglycans (FIGS. 21G-J). A continuous increase in ECM production was observed between 7 days and 14 days, with the encapsulated cells displaying a spherical morphology and lacuna structures, the hallmarks of chondrocyte phenotype during chondrogenesis. These findings indicate that the NC-PEG-ALG bioscaffold effectively supports MSC growth and chondrogenic differentiation.

As described above, the developed composite bioink, which includes defined concentrations of NC, PEG, and ALG, has an exceptional compressive strength and a Young's modulus similar to that of the articular cartilage tissue. The bioink's unique mechanical properties enhance cartilage repair by replicating the microenvironment of chondrogenic progenitors, supporting extracellular matrix production and maintaining cartilage phenotypes. Its enhanced printability enables the fabrication of 3D bioprinted cartilage organoids that mimic the anatomical structure of cartilage, providing a biomimetic environment for encapsulated chondrogenic cells to proliferate and differentiate. These encapsulated specialized chondrogenic cells produce essential extracellular matrix (ECM) components, such as type II collagen and glycosaminoglycans, ultimately forming functional artificial cartilage for drug development, disease modeling, and the repair of osteoarthritic defects or articular cartilage lesions.

The values of compressive breaking stress, Young's modulus, and viscosity cited in the present invention represent the average of three tested samples. For breaking strength of NC-PEG-ALG, the recorded minimum value was 21.2 MPa, while the maximum value was 37.8 MPa, resulting in a range of 21.2-37.8 MPa. Likewise, the measured Young's modulus for NC-PEG-ALG ranged from 137.7 to 182.65 kPa. The viscosity measurements fell within the range of 2867.0-2928.0 Pa·s. Tables 2 and 3 listed the comparison between minimum and maximum values of the breaking strength and the comparison between different compositions of ingredients used in the present invention respectively as well as a comparison in viscosity.

TABLE 2

| Breaking strength (MPa) | Maximum value | Minimum value |
|---|---|---|
| NC-PEG-ALG | 37.8 | 21.2 |

TABLE 3

| Multiple comparison | P-value |
|---|---|
| ALG vs. NC-PEG-ALG | 0.0006 |
| ALG vs. PEG-ALG | 0.0020 |
| NC-PEG-ALG vs. PEG-ALG | 0.044 |

TABLE 4

| Viscosity (Pa · s) | Maximum value | Minimum value |
|---|---|---|
| NC-PEG-ALG | 2928.0 | 2867.0 |

The composite bioink is easy to extrude and bioprint, enabling the creation of highly customized, biomimetic cartilage organoids. 3D cartilage organoids are generated by extrusion plotting methods and exhibit native cartilage phenotypes. These 3D cartilage organoids are precisely tailored accordingly to patient-specific information obtained from medical imaging, offering promising printing fidelity for personalized therapies, drug development and disease modelling.

Moreover, the incorporation of ALG and mineral clay is anticipated to increase the Young's modulus and the breaking stress of the material by forming a more rigid microstructure and fostering stronger interactions between polymer chains and monodispersed clay particles. This enhances structural integrity and supports better 3D cell aggregation during culture, delivering mechanical stimuli that more closely mimic the native cartilage extracellular matrix (ECM).

Additionally, the clay's presence has great potential to overcome PEG's bioinert nature by absorbing bioactive molecules and regulating their release, thereby improving the biological functionality of the composite material. The resulting cartilage organoid can precisely replicate the anatomical structure and biological functions of native joint cartilage.

This approach is believed to offer substantial advantages over treatments, providing a novel solution for articular cartilage restoration. Cartilage tissue engineering typically involves isolating a small number of cartilage-seed cells from a donor, expanding them in culture, and then seeding them onto biocompatible scaffolds. These scaffolds serve as graft substitutes and are implanted into cartilage defects. As the scaffold degrades, the seeded cells proliferate and produce the cartilage matrix, ultimately forming new cartilage tissue. The success of this process relies on the careful selection of biomaterials and seed cells to ensure effective cartilage regeneration.

The present invention explained above is not limited to the aforementioned embodiment and drawings, and it will be obvious to those having an ordinary skill in the art of the prevent invention that various replacements, deformations, and changes may be made without departing from the scope of the invention.

1. A method for generating three-dimensional (3D) cartilage organoids through bioprinting, the method comprising:
expanding mesenchymal stem cells (MSCs);
preparing a composite bioink; and,
bioprinting a cell-laden scaffold to generate the 3D cartilage organoids.

2. The method of claim 1, wherein the step of expanding the mesenchymal stem cells (MSCs) further comprising:
obtaining human umbilical cord cell suspension in-vitro as a source of the MSCs;
culturing the human umbilical cord cell suspension in an optimum culturing environment; and,
subculturing the human umbilical cord with a new culture medium to obtain a monolayer culture of human umbilical cord-derived MSCs; and,
replacing the culture medium at regular intervals until a desired cell density is achieved.

3. The method of claim 1, wherein the step of preparing the composite bioink further comprising:
mixing sterilized water and clay, forming a water-clay mixture;
dissolving a functionalized polymer and alginate powders into the water-clay-mixture forming a hydrogel solution; and,
sterilizing the hydrogel solution to obtain purified composite bioink.

4. The method of claim 1, wherein the step of bioprinting the cell-laden scaffold to generate the 3D cartilage organoids further comprising:
detaching and transferring a monolayer culture of the human umbilical cord-derived MSCs into a purified composite bioink to form a cell-laden bioink;
thoroughly mixing the cell-laden bioink;
removing residual bubbles by centrifuging the mixed cell-laden bioink;
transferring the mixed cell-laden bioink into a 3D bioprinter with pre-determined parameters to fabricate a 3D cell-laden scaffold in the form of cubic blocks through cross-linking; and
culturing the 3D cell-laden scaffold in an inductive culture medium to generate the 3D cartilage organoids.

5. A three-dimensional (3D) cartilage organoid for use in disease modeling and screening, the organoid comprising:
human multipotent mesenchymal stem cells (MSCs) and a composite bioink.

6. The 3D cartilage organoid of claim 5, wherein the MSCs proliferate and differentiate into progenitor or specialized chondrogenic cells, supporting tissue development and regeneration.

7. The 3D cartilage organoid of claim 5, the composite bioink is a mixture of sterilized water, 6±0.1% w/v clay, 12±0.1% w/v functionalized polymer and 6±0.1% w/v alginate powders.

8. The 3D cartilage organoid of claim 7, wherein the functionalized polymer includes but is not limited to polyethylene glycol (PEG) modified with vinyl sulfone (VS) groups, configured to enable thiol-based crosslinking.

9. The 3D cartilage of claim 7, wherein the clay includes but is not limited to nanoclay.

10. The 3D cartilage of claim 5, wherein the organoid includes but is not limited to a patient-specific model that replicates the anatomical structure of native cartilage to improve disease modeling and screening outcomes.

11. The 3D cartilage organoid of claim 5, wherein the organoid has achieved a Young's modulus within a range of 137.7-182.65 kPa with a viscosity within a range of 2867.0-2928.0 Pa·s.

12. The 3D cartilage organoid of claim 5, wherein the organoid has a breaking stress within a range of 21.2-37.8 MPa.
